(19) United States
(12) Patent Application Publication
(10) Pub. No.: US 2017/0248516 A1
(43) Pub. Date: Aug. 31, 2017

(54) CALIBRATION DEVICE

(71) Applicants: DIAMED GMBH, CRESSIER (CH); CSEM Centre Suisse d'Electronique et de Microtechnique SA - Recherche et Development, NEUCHATEL (CH)

(72) Inventors: GERMANO MEIER, FRIBOURG (CH); RAPHAEL PUGIN, AUVERNIER (CH); DAVID HASLER, NEUCHATEL (CH); EMMANUEL SCOLAN, NEUCHATEL (CH)

(21) Appl. No.: 15/315,517

(22) PCT Filed: Jun. 2, 2014
(86) PCT No.: PCT/EP2014/061395

§ 371 Date: Dec. 1, 2016

(51) Int. Cl. G01N 21/27; A61B 5/00; H04N 1/60; H04N 17/00; G06T 7/80

(52) U.S. Cl. CPC G01N 21/278; H04N 17/002; G06T 7/80; H04N 1/6033; A61B 5/0059; A61B 2560/0223

(57) ABSTRACT

Calibration device for a diagnostic device, the diagnostic device comprising a digital imaging device arranged to record an image of at least one sample container containing at least one biological sample. According to the invention, methods include of digitally imaging said at least one sample container, thus obtaining a first image processing the first image, thus obtaining a second image printing a representation of the second image onto a substrate by means of a printing method, thus obtaining the calibration device, wherein said processing of the first image comprises applying at least one transfer function to the first image. The invention further relates to a corresponding calibration device, and a method of calibrating a diagnostic device by means of the calibration device.

### TECHNICAL FIELD

The present invention relates to the field of diagnostic laboratory equipment. More particularly, it relates to calibration devices for the calibration of such diagnostic devices.

### STATE OF THE ART

Diagnostic laboratory equipment for automated diagnosis must be calibrated periodically to ensure their accuracy. Recent EU regulation requires that such diagnostic devices be calibrated using a standardised and reproducible procedure. As an example, in the case of blood analysis, the diagnostic device is typically calibrated using real blood samples with known properties (e.g. blood type, presence of pathogens), to determine whether the diagnostic device correctly diagnoses these known properties. If not, the diagnostic device is adjusted appropriately, or sent for repair. Typically, a blood sample of known properties is introduced into a container which contains a reagent, with which the blood sample reacts to a greater or lesser extent, generating a pattern characteristic of the property being tested. The container may be one of several provided in a so-called diagnostic bio-card, each container comprising a different reagent for determining different properties of the sample. An image of the container is created using a digital camera integrated in the diagnostic device, and the image thus obtained is then processed using an ad hoc algorithm, adapted to produce a diagnostic about the biological sample, e.g. the presence of an antigen, blood group, presence of pathogens, etc.

Since real biological samples are used for calibration, the reaction of the blood with the reagent is unstable over time. In consequence, the shelf life of the calibration samples is limited to several hours, requiring new calibration samples to be produced every time the diagnostic laboratory equipment must be calibrated, which furthermore requires a stock of appropriate biological material to be maintained. In consequence, such calibration procedures are expensive and time-consuming.

Alternative calibration methods use for instance colour cards, optical filters and so on for calibration. Although these methods can calibrate the camera parameters, such as focus, colour response, etc. of the diagnostic device, they cannot simulate real reactions, 3D effects and camera limitations (such as fisheye, undesirable borders etc.) and thus cannot be used for calibrating the diagnostics performed by the diagnostic device.

The object of the invention is thus to overcome at least partially the above-mentioned disadvantages of the prior art.

### DISCLOSURE OF THE INVENTION

The object of the invention is attained by a method for manufacturing a calibration device for a diagnostic device, the diagnostic device comprising a digital imaging device arranged to record an image of at least one sample container containing at least one biological sample, which may be for instance a blood sample, urine sample, or other tissue sample that may or may not have been exposed to a reagent. According to the invention, the method comprises digitally imaging at least one sample container containing at least one biological sample of known properties, thus obtaining a first image. This first image is then digitally processed, thus obtaining a second image. Subsequently, a representation of the second image is printed onto a substrate by means of a printing method, thus obtaining the calibration device. The processing of the first image comprises applying at least one transfer function to the first image.

The first image can be either be a single image recorded of a single original sample as mentioned above, or be generated by combining several such first images as part of applying the transfer function. For example, the image may be a patchwork of several versions of first images stemming from different biological samples. It could also be generated by imaging several times the same biological sample and combining the results so as to obtain an image with less noise and/or better resolution. Furthermore, for rare diagnostics for which a real sample is difficult to obtain, the transfer function may furthermore comprise a manipulation of the first image so as to simulate the rare diagnostic response.

In consequence, the thus-obtained calibration device can be used for calibration of the diagnostic device in the place of actual biological samples of known properties. Since printed material has a usable shelflife of 5-10 years or even longer, repeated preparation of samples for calibration is thus no longer necessary, and the repeatability of the calibration is assured. Furthermore, since the calibration device can be duplicated, the same type of calibration device can be used as standard for all diagnostic devices of a particular model. Compared to prior art calibration methods not using a real sample, the thus-obtained calibration device can simulate real reactions, 3D effects and cameral limitations (such as fisheye, undesirable borders etc.)

In an embodiment, the transfer function is calculated such that a third image obtained by digitally imaging the calibration device with the digital imaging device of the diagnostic device substantially corresponds to an image of said at least one sample container obtained by digitally imaging said at least one sample container with the digital imaging device of the diagnostic device. Thus, any distortion of the image or the colour thereof due to the printing method can be compensated for.

In an embodiment, at least part of said transfer function is computed by printing a testchart with known colour values onto a substrate by means of said printing method, taking an image of the testchart with the digital imaging device of the diagnostic device, thereby obtaining a fourth image, and comparing the colour values of the fourth image with the known colour values of the testchart. By “colour values”, we mean the Red-Green-Blue code used to represent the colour. The colour distortion due to the printing method can thus be compensated over a wide colour gamut by measuring this distortion accurately and directly.

In an embodiment, the first image is obtained by the digital imaging device of the diagnostic device, providing simple, direct obtention of the first image, with the correct optical arrangement. Alternatively, the first image is obtained by a further digital imaging device of substantially identical optics to, and higher resolution than, the digital imaging device of the diagnostic device, which enables a higher resolution and thus more accurate representation of the at least one biological sample of known properties to be produced and printed on the calibration device. In this latter case, said at least one transfer function further comprises a transfer function compensating for the difference between the further digital imaging device and the digital imaging device of the diagnostic device. Thus, any difference in the colour rendition between the further digital imaging device and the digital imaging device of the diagnostic device can be compensated.

In an embodiment, the printing method further comprises applying an adhesive layer and a substantially transparent sealing layer to the substrate, thereby encapsulating the representation of the second image printed onto the substrate. The ink is thus protected from atmospheric oxygen and other chemicals, reducing degradation and fading thereof, and ensuring thereby a long service life of the calibration device.

In an embodiment, the representation of the second image is printed onto said substrate in colour.

The object of the invention is likewise attained by a calibration device for calibration of a diagnostic device, the diagnostic device comprising a digital imaging device arranged to record an image of at least one biological sample, the calibration device having a structure comprising at least a substrate and a printed layer printed on the substrate.

According to the invention, the printed layer comprises a printed representation of at least one sample container containing at least one biological sample of known properties such as a blood sample, urine sample, or any other biological sample whether having been exposed to a reagent or not. The printed representation is arranged such that an image of the calibration device taken with the digital imaging device of the diagnostic device substantially corresponds to an image of said at least one sample container obtained by digitally imaging said at least one sample container containing a sample of known properties with the digital imaging device of the diagnostic device.

In consequence, the calibration device can be used for calibration of the diagnostic device in the place of actual biological samples of known properties as discussed above. Thus, this calibration can also be done on diagnostic device that was not designed to perform such a calibration.

Advantageously, the calibration device further comprises an adhesive layer applied on to at least the printed layer, and a substantially transparent sealing layer applied on to the adhesive layer. The ink is thus protected from atmospheric oxygen and other chemicals, reducing degradation and fading thereof, and ensuring thereby a long service life of the calibration device.

In an embodiment of the calibration device, the substrate is a substantially planar substrate, providing simple and cheap construction. Alternatively, the substrate may be three-dimensional, providing greater realism to the calibration device.

Advantageously, the calibration device further comprises a three-dimensional part provided on said structure, enabling handling by automated diagnostic devices.

Preferably, the substrate and/or the sealing layer each are transparent, opaque, or translucent.

Finally, the object of the invention is attained by a method for calibrating a diagnostic device, the diagnostic device comprising a digital imaging device arranged to record an image of at least one biological sample, comprising providing a calibration device as described above, imaging the calibration device with the digital imaging device of the diagnostic device, thus obtaining a calibration image, and comparing the calibration image with said at least one biological sample of known properties. Naturally, the original biological sample does not need to be present, since its properties are already known.

Thus, the necessity to use genuine biological samples for calibration of the diagnostic device is eliminated.

Preferably, the step of comparing the calibration image with said at least one biological sample of known properties comprises performing diagnostics on the calibration image to determine whether said known properties are correctly diagnosed.

The step of comparing the calibration image with said at least one biological sample of known properties may also comprise comparing the calibration image with said image of said at least one sample container, for instance by comparing the colours of the images.

It is to be noted that some of the older diagnostic devices perform the diagnostic based on a grayscale version of the image. For such devices, the calibration device may be printed in grayscale, and the transfer functions applied to the image are one dimensional, thus easily computable. On those equipments, the camera might be a grayscale camera, but can also be a colour camera. In either case, printing can be performed in grayscale or in colour. In this situation, the printed representation is arranged such that the grayscale version of an image of the calibration device taken with the digital imaging device of the diagnostic device substantially corresponds to the grayscale version of an image of said at least one sample container obtained by digitally imaging said at least one sample container containing a sample of known properties with the digital imaging device of the diagnostic device. Note that if the colour versions of the image match, the grayscale version also matches, but the contrary is not necessarily true.

On more recent diagnostic devices, the analysis is performed in colour space, and it is of fundamental importance to accurately reproduce colour. Not only the transfer functions must be computed to accurately reproduce the colour, but the printing equipment, that is, the printer-substrate combination must be chosen such that any colour that might be observed on the biological sample must be reproducible on the printed sample, in other words, the gamut of the printing system must contain the gamut of the biological sample, considering the gamut after transformation by the diagnostic device camera.

### BRIEF DESCRIPTION OF THE DRAWINGS

Further details of the invention will now be explained in reference to the following figures, which show:

FIG. 1: a schematic representation of a method of manufacturing a calibration device;

FIG. 2: a schematic representation of a method of calibrating a diagnostic device;

FIG. 3: a schematic representation of an embodiment of the structure of a calibration device;

FIG. 4: a representation of a calibration device provided with a three-dimensional part.

### EMBODIMENT OF THE INVENTION

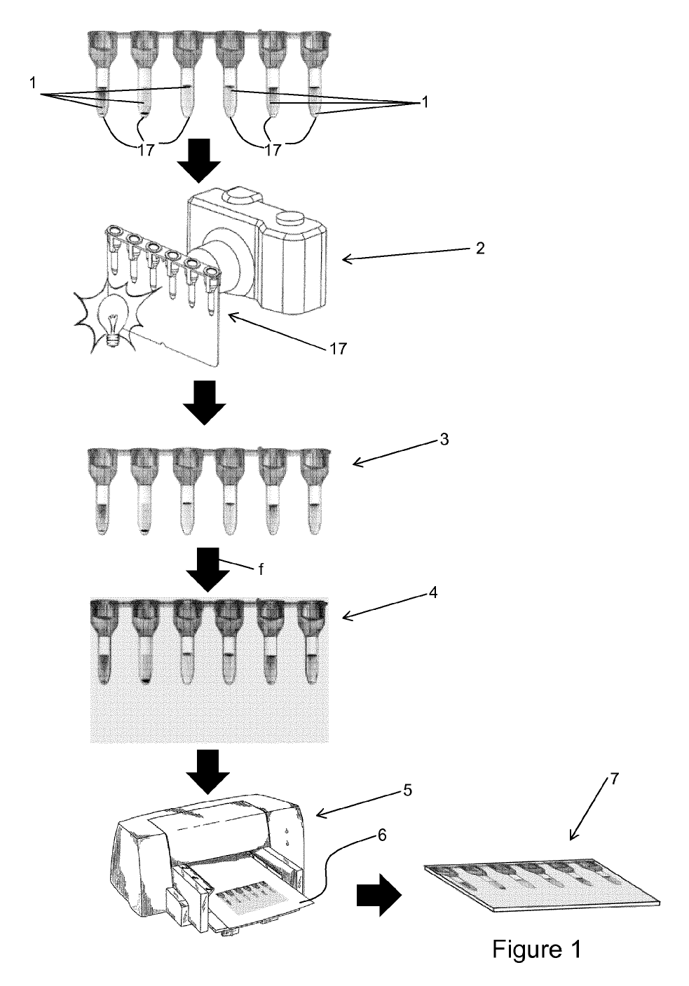

FIG. 1 illustrates schematically a method of manufacturing a calibration device according to the invention.

Firstly, at least one biological sample 1 of known properties is introduced into at least one sample container 17, as is conventionally known. In this case, a bio-card of six sample containers 17 is illustrated. This biological sample 1 may e.g. comprise one or more blood samples of known blood group, treated with appropriate reagents. These reagents may cause coagulation, colour changes, and/or floating or settling out to varying degrees of the solid reaction products. These changes are typically determinant for diagnosing the known properties of the biological samples.

Subsequently, the sample containers 17 containing the biological samples 1 are digitally imaged, either by utilising a digital imaging device 2 such as a digital camera of a diagnostic device of the type for which the calibration device is intended, or by a separate digital camera. This imaging step results in a first image 3, which is a raw image of the sample containers 17.

The first image 1 is then processed by applying a transfer function f thereto as will be described below, thereby generating a second image 4.

Second image 4 is then printed onto a substrate 6 utilising a printing method, represented schematically by printer 5, thus obtaining calibration device 7 in its most basic form. Substrate 6 may be, for instance, paper, card, transparent film, or a three-dimensional substrate, and the printing method may be an inkjet, laser-printing, lithographic, or any other convenient method.

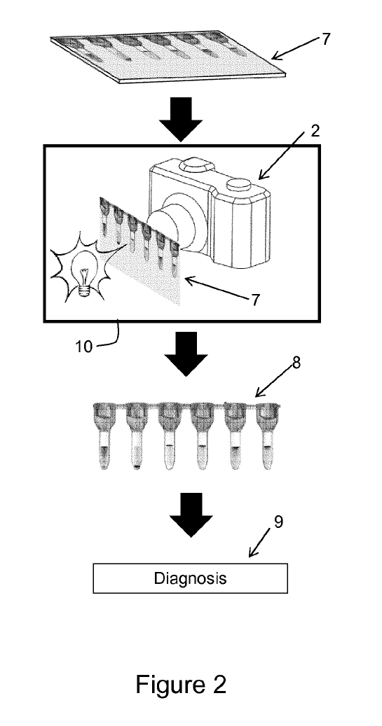

FIG. 2 illustrates a method of calibrating a diagnostic device according to the invention. Firstly, a calibration device 7 as described above is provided, which is then imaged with a digital imaging device 2 of a diagnostic device 10, thereby obtaining a calibration image 8. A diagnosis 9 is carried out upon the calibration image 8, to determine whether the known properties of the original biological samples 1 are correctly diagnosed. If not, the diagnostic device must be adjusted, or sent for repair. In short, the calibration device 7 of the invention merely replaces the actual biological samples for calibration of the diagnostic device.

The diagnosis 9 may alternatively or additionally comprise comparing the calibration image 8 with the first image 3. It allows to slightly adjust the calibration device to compensate a slight drift in its lighting or imaging hardware, or just to measure the drift from a photometric point of view rather than from a biological diagnostic point of view. In this case, a testchart with known colour values is preferably superimposed to the first image 3 in an area that does not contain any representation of a biological sample. The testchart eases the reading of a particular colour value for comparison with the desired output stemming from the first image 3.

Typically, the diagnostic device is arranged to generate a result among N possible results. If the calibration device comprises representations of clear examples of the known properties to be calibrated among the N possible results, the calibration is binary: either the diagnostic device correctly diagnoses each sample, or fails for one or more possible results. Advantageously, the calibration device can furthermore comprise representations of biological samples that are close to the decision boundary, i.e. that exhibit a property less strongly, and thus could possibly be diagnosed in more than one of the N classes. By using such a representation close to the decision boundary, or by using several representations of properties crossing the decision boundary, a slight degradation in the performance of the diagnostic device can be identified, since the properties of the original biological samples will be misdiagnosed first for the representations close to the decision boundary.

Alternatively, to detect a slight degradation of the diagnostic device, an intermediate representation of the diagnostic may be used. Typically, a diagnostic device is computing one or several parameters from the image, each resulting in a score. These scores are then categorised into said N classes. Instead of comparing the categorised score resulting from processing the image 8 from the calibration device, the calibration method may compare the score computed from first image 3 or calibration image 8 at the time when the diagnostic device is running according to specifications, with the scores computed from image 8 at a later time. In this way, the degradation of the diagnostic device can be detected before it gets to the stage in which misinterpretation of a calibration device 7 occurs. Since misinterpretation of calibration device 7 is thereby prevented, the diagnostic device still functions according to specifications, and thus can continue to be used in its daily function, but thanks to the detection of the degradation, a maintenance procedure can be scheduled on that device, and proper actions can be taken to avoid interrupting the business that relies on the diagnostic device.

It should thus be understood that calibration image 8 should substantially correspond to first image 3, within the system limitations and system tolerances. Since the printing method and indeed the material of the substrate 6 and any sealing layers (see below) applied thereto may alter the colours of the first image, this must be taken into account by applying the above-mentioned transfer function f to the first image so as to obtain the second image which, when printed and subsequently imaged by the digital imaging device 2 of the diagnostic device, will give a calibration image 8 substantially corresponding to first image 3.

Transfer function f can be calculated by printing a colour test chart comprising a set of colour patches with known RGB values (Cc) using the same above-mentioned printing method. These known RGB values are modified by the printing method and the materials of the substrate 6, and this modification can be determined by imaging the colour test chart in the diagnostic device 10, and comparing each of the measured RGB values (Ct) of the image of the colour test chart with the corresponding known RGB values. Such measurements can be effected using software that generates ICC profiles. From this comparison, a transfer function g: Cc [symbol] Ct can be calculated, which can be inverted to give transfer function f, which can also be expressed as f: Ct [symbol] Cc. To compute function f, two main steps are involved: The first step comprises selecting every colour value Cti of a regular and complete RGB grid, convert it to a given colour space, for example Lab, project this colour on the measured gamut, i.e. the envelope in said colour space defined by the totality of all Ct values, and obtaining colour Ctip. This step is called “gamut mapping”, and does only modify value Cti if Cti is outside said colour gamut. The second step comprises selecting the neighbouring colours of Ctip in the set of Ct measurements—expressed in said colour space—find a linear combination of Ct values that are approximately equal to value Ctip, and apply this linear combination to the values Cc that correspond to selected colours Ct, and obtain a value Cci. Transfer function f is embodied as a set of values f:Cti [symbol] Cci. In the process of computing said linear combinations, a smoothness constraint may be applied to function f:Cti [symbol] Cci. Transfer function f can be applied to the first image 3 e.g. in a commercial image editing package such as Adobe Photoshop®, so as to generate the second image 4. It should be further noted that, when calculating the transfer function g, the printed colour test chart should be printed in exactly the same manner as the calibration device 7, and the ink should be allowed to dry and stabilise before the RGB values of the test chart are measured.

In the case in which the resulting second image is of an unacceptably low resolution e.g.

due to lowpass filtering caused by both the lens and sensor of the digital imaging device 2 when producing the first image 3, resulting in an unacceptably low resolution calibration image 8 when the calibration device 7 is imaged by the lens and sensor of the digital imaging device 2 of the diagnostic device 10 thereby again being subjected to further lowpass filtering, the digital imaging device used for generating the first image 3 may have a higher resolution than the digital imaging device 2 of the diagnostic device 10. Such a higher resolution digital imaging device should ideally have optics exhibiting the same geometric projection properties as the digital imaging device 2 of the diagnostic device 10, but a higher resolution. Since the two digital imaging devices will not be identical, a transfer function h between the two digital imaging devices 2 must be calculated, e.g. by comparing RGB values of images of a colour test chart imaged by the higher resolution digital imaging device and of the same colour test chart imaged by digital imaging device of the diagnostic device 10. The inverse of function h must in consequence be applied to the said first image 3 taken by the higher resolution digital imaging device when calculating transfer function f above.

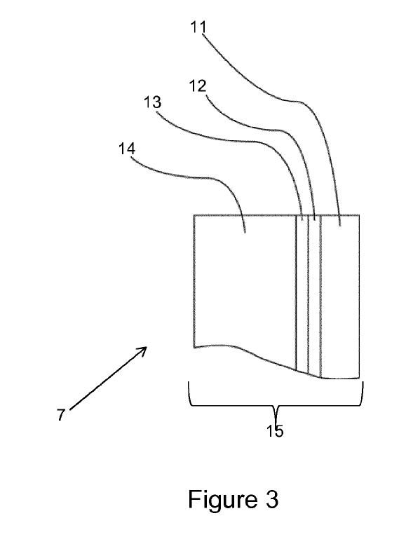

FIG. 3 illustrates schematically an embodiment of the cross-sectional structure of a calibration device 7 according to the invention.

In the embodiment illustrated in FIG. 3, calibration device 7 comprises a structure 15, which in the present example is a sandwich structure. This structure comprises a substrate 11, upon which an ink layer 12 constituting a printed representation of the second image 4 is deposited. The substrate 11 may be for instance a 100 μm PET foil, which exhibits excellent transparency, excellent thermal and dimensional stability, and excellent ageing properties. This PET foil may be, for instance, coated with a substantially crack and defect free SiO₂-based nano-porous layer, which exhibits excellent printing stability, transparency and resolution. This silica nanoporous layer is ideally composed of nanoparticles with a diameter ranging between 5 and 20 nm and a polymeric binder, preferably polyvinyl-alcohol with content in the 10-25 wt % range compared to silica. The dry thickness is ideally lower than 60 μm and preferably in the 10-40 μm range. This kind of silica nanoporous layers is fully compatible with pigmented inks which provide higher resolution and stability than fully dyed ones. For the printing, an Epson Stylus Pro® R3880 inkjet printer using pigment-based inks has proven suitable, since this printer exhibits a gamut large enough to print the required colours with the required degree of accuracy. The ink layer 12 is applied to the nano-porous layer, and after the ink has dried, and considered as part of the printing method, an adhesive layer 13 is applied, and a sealing layer 14 is applied thereto, e.g. using standard lamination equipment. Sealing layer 14 may, for instance, be a plastic card such as a sheet of polycarbonate, lending structural stability to the calibration device 7. Alternatively, glass may be used for sealing layer 14. However, if the substrate 11 exhibits sufficient structural stability on its own, sealing layer 14 may simply be a thin polymer foil.

In the case of a non-transparent calibration device 7, the ink layer 12 may be printed on matt paper or an opaque foil, and an appropriate sealing layer 14 may be adhered thereto.

Since the ink layer 12 is sealed by adhesive layer 13 and sealing layer 14, it is not exposed to oxygen or other chemicals which may accelerate deterioration and fading of the pigments in the ink. Ideally, an adhesive should be chosen for the adhesive layer 13 that does not interact with the pigments of the ink layer 12, to further avoid deterioration of the ink.

However, it should be noted that the structure of FIG. 3 is merely one example of a possible structure. If an ink with sufficient oxygen stability can be utilised, adhesive layer 13 and sealing layer 14 may be dispensed with entirely, and the ink layer 12 may be applied directly onto a transparent or nontransparent substrate 11, such as the above-mentioned PET foil, paper, card, or any other convenient material presenting the required printing properties.

Alternatively, substrate 11 may comprise three-dimensional structure, e.g. at least partially reflecting the shape of the at least one sample container 17. For instance, the ink layer 12 may be printed onto a flat surface of the substrate, the other surface being 3-D structured representing the profile of half of the thickness of the at least one sample container 17. Or, the ink layer 12 may be printed onto a 3-D structured substrate representing the entire profile of the at least one sample container 17.

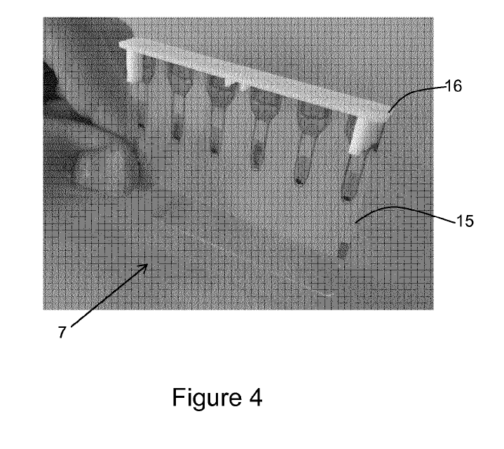

Advantageously, the calibration device 7 may comprise an additional three-dimensional part 16 attached thereto, e.g. by adhesive, form-fitting, rivets, clips or similar, to an edge of the structure 15, as shown in FIG. 4. This three-dimensional part 16 is formed substantially as a flange, arranged substantially perpendicular to the plane of the structure 15, and assists in the handling of the calibration device 7 by automated diagnostic devices, by replicating the form of the equivalent part of the real sample containers 17 that the automated handling system manipulates. Three-dimensional part 16 may further comprise a protective aluminium foil for automatic identification and position detection.

Although the invention has been described in terms are specific embodiments, variations thereto are possible for the skilled person without departing from the scope of the invention as defined by the appended claims.

1-17. (canceled)

18. A method for manufacturing a calibration device for a diagnostic device comprising a digital imaging device arranged to record an image of at least one sample container, the method comprising the steps of:
digitally imaging at least one sample container containing at least one biological sample to obtain a first image;
processing the first image to obtain a second image, wherein the processing of the first image comprises applying at least one transfer function to the first image; and
printing a representation of the second image onto a substrate to obtain a calibration device.

19. The method of claim 18, further comprising:
calculating the at least one transfer function such that a third image obtained by digitally imaging the calibration device with the digital imaging device of the diagnostic device substantially corresponds to an image of said at least one sample container obtained by digitally imaging said at least one sample container with the digital imaging device of the diagnostic device.

20. The method of claim 18, further comprising:
computing at least a part of the at least one transfer function by:
printing a testchart with known color values onto a test substrate;
taking an image of the testchart with the digital imaging device of the diagnostic device, thereby obtaining a fourth image; and
comparing the color values of the fourth image with the known color values of the testchart.

21. The method of claim 18, further comprising:
obtaining the first image via the digital imaging device of the diagnostic device.

22. The method of claim 20, further comprising:
obtaining the first image by a further digital imaging device that has substantially identical optics and a higher resolution relative to the digital imaging device of the diagnostic device.

23. The method of claim 22, wherein said at least one transfer function comprises a transfer function compensating for a difference between the further digital imaging device and the digital imaging device of the diagnostic device.

24. The method of claim 18, further comprising:
applying an adhesive layer and a substantially transparent sealing layer to the substrate, thereby encapsulating the representation of the second image printed onto the substrate.

25. The method of claim 18, wherein the step of printing the representation of the second image onto the substrate comprises printing the representation of the second image onto said substrate in color.

26. A calibration device for calibration of a diagnostic device that comprises a digital imaging device arranged to record an image of at least one biological sample, the calibration device comprising:
a substrate;
a printed layer printed on the substrate; and
the printed layer comprising a printed representation of at least one sample container that contains at least one biological sample of known properties; and
wherein said printed representation is arranged on the substrate such that an image of the calibration device taken with the digital imaging device of the diagnostic device substantially corresponds to another image of said at least one sample container that is obtained by digitally imaging said at least one sample container with the digital imaging device of the diagnostic device.

27. The Calibration device according to claim 26, further comprising:
an adhesive layer applied onto at least the printed layer; and
a substantially transparent sealing layer applied onto the adhesive layer.

28. The Calibration device according to claim 26, wherein the substrate is a substantially planar substrate.

29. The Calibration device according to claim 26, wherein the substrate is three-dimensional.

30. The Calibration device according to claim 26, wherein at least one of the sealing layer and the substrate is a state chosen from the group consisting of transparent, opaque and translucent.

31. The Calibration device according to claim 26, further comprising:
at least one three-dimensional part arranged on an edge of the device.

32. The Calibration device according to claim 26, wherein said substrate comprises a nanoporous silica layer, the nanoporous layer comprising an attribute selected from the group consisting of:
comprising nanoparticles having a diameter ranging between 5 and 20 nm;
comprising a polymeric binder that has a weight ranging from 10% to 25% of a weight of a comparable silica polymeric binder; and
comprising a dry thickness lower than 60 μm

33. The Calibration device according to claim 32, wherein the polymeric is polyvinyl-alcohol.

34. The Calibration device according to claim 32, wherein the dry thickness is between 10 μm and 40 μm.

35. A Method for calibrating a diagnostic device, the diagnostic device comprising a digital imaging device arranged to record an image of at least one biological sample, the method comprising the steps of:
printing a layer on a substrate, wherein the printed layer comprises a printed representation of at least one sample container that contains at least one biological sample of known properties, and wherein said printed representation is arranged on the substrate such that an image of a calibration device taken with a digital imaging device of a diagnostic device substantially corresponds to another image of said at least one sample container that is obtained by digitally imaging said at least one sample container with the digital imaging device of the diagnostic device;
imaging the calibration device with the digital imaging device of the diagnostic device, thus obtaining a calibration image;
comparing the calibration image with said at least one biological sample of known properties.

36. The method of claim 35, wherein the step of comparing the calibration image with said at least one biological sample of known properties comprises a step selected from the group consisting of:
comparing the calibration image with said image of said at least one sample container containing at least one biological sample of known properties; and
performing diagnostics on the calibration image to determine whether said known properties are correctly diagnosed.
